(19) United States
(12) Patent Application Publication
(10) Pub. No.: US 2008/0220052 A1
(43) Pub. Date: Sep. 11, 2008

(54) Body weight gain inhibitor

(75) Inventors: Takahiro Tsujita, Toon-shi (JP); Takeshi Takaku, Toon-shi (JP); Naoyuki Yoshida, Ichihara-shi (JP)

Correspondence Address: HOGAN & HARTSON LLP;IP GROUP, COLUMBIA SQUARE, 555 THIRTEENTH STREET, N.W., WASHINGTON, DC, 20004, US

(73) Assignees: EHIME UNIVERSITY; CHISSO CORPORATION

(21) Appl. No.: 12/149,674

(22) Filed: May 6, 2008

Related U.S. Application Data
(62) Division of application No. 11/476,942, filed on Jun. 29, 2006.

(30) Foreign Application Priority Data
Jun. 30, 2005 (JP) 2005-191104
Feb. 15, 2006 (JP) 2006-037775

(51) Int. Cl. A61K 9/68; A61K 38/02; A61K 9/00

(52) U.S. Cl. 424/439; 514/2; 424/440

(57) ABSTRACT

The invention provides a body weight gain inhibitor, a fat amount gain inhibitor, a medicament for prevention and/or treatment of obesity, a food composition having inhibitory effect on body weight gain and a food composition having inhibitory effect on fat amount gain, each containing ε-polylysine or a salt thereof. In another aspect of the invention, the described compositions are useful for inhibiting body weight gain, inhibiting fat amount gain and preventing and/or treating obesity.

### CROSS REFERENCE TO RELATED APPLICATIONS

This application claims priority under 35 U.S.C. § 119 to Japanese Patent Application No. JP 2005-191104, filed Jun. 30, 2005, and Japanese Patent Application No. 2006-37775, filed Feb. 15, 2006, which applications are expressly incorporated herein by reference in their entirety.

### BACKGROUND OF THE INVENTION

1. Field of the Invention

The invention relates to a body weight gain inhibitor and food compositions having an inhibitory effect on body weight gain, etc. Specifically, the invention relates to a body weight gain inhibitor or food compositions having an inhibitory effect on body weight gain, etc., each containing ε-polylysine.

2. Description of the Related Art

Obesity is a risk factor for developing lifestyle diseases such as diabetes, hypertension and hyperlipemia. Therefore, preventing body weight gain is considered to be important for preventing the incidence of lifestyle diseases.

It is widely recognized that improving lifestyle habit, for example, by improving the food content of meals or taking sufficient exercise, is important for preventing body weight gain. In practice, however, it is not easy to change lifestyle habits, and in many cases, only changing lifestyle habit is not sufficient for preventing body weight gain. Accordingly, medical agents and foods which inhibit obesity and weight gain are strongly desired.

At present, sibutramine, which is a catecholamine-serotonin reuptake inhibitor, and rimonabanto, which is a CB1 receptor inhibitor, are known as anti-obesity drugs. Further, orlistat is known as an inhibitor for pancreatic lipase which takes part in fat metabolism.

Meanwhile, ε-polylysine is known as a food additive having an antibacterial effect (refer to, for example, Japanese Kokai Publication No. 2003-171462). In a report regarding the safety of ε-polylysine by Ishii or Hiraki (see, e.g., The Clinical Report (Kiso to Rinsho) 27, 2013-2033 (1993) and Regulatory Toxicology and Pharmacology 37, 328-340 (2003)), it is reported that the intake of a high concentration of ε-polylysine causes inhibition of body weight even without eating high fat food, and it is considered to be one factor of the body weight inhibiting effect that has some influence on test substances on the amount of food intake of test animals according to food faddiness (i.e., the likes and dislikes of food).

Further, in recent years, knowledge regarding involvement of fat absorption inhibition by inhibiting pancreatic lipase reaction is obtained as a new function of ε-polylysine. That is, ingested fat forms an emulsion between the stomach and the duodena in the presence of bile acid and then is acted on by pancreatic lipase. The emulsion is turned into a micelle form by the above action of pancreatic lipase, and it is absorbed from the bowel. However, the reaction of pancreatic lipase is inhibited by ε-polylysine present on the surface of the emulsion if the ε-polylysine is present at the time of the formation of the emulsion. It is believed that because the emulsion containing a fat becomes hard to be converted into a micelle, absorption of a fat from an intestinal wall is inhibited. For example, Tsujita et al. reported a fundamental mechanism regarding inhibition of pancreatic lipase by ε-polylysine (see, e.g. J. Lipid. Res. 44, 2278-2286 (2003)). Similarly, Kido et al. reported that decrease in blood neutral fat of rats ingesting a high fat food takes place depending on a dose of ε-polylysine (see, e.g., J. Nutr. 133, 1887-1891 (2003)). Also, regarding ε-polylysine, the action of inhibiting increase of serum cholesterol level and liver cholesterol level is reported (see, e.g., Japanese Kokai Publication No. Hei 4-221320). Further, it is reported that absorption of fat can be disturbed or inhibited by disturbing or inhibiting the action of a lipid digesting enzyme by ε-polylysine (see, e.g., Japanese Kokai Publication No. Hei 3-284627).

However, an inhibition in weight gain by practical administration or intake of ε-polylysine which leads to an improvement in weight gain and obesity due to a dietary life centered on fatty foods has not so far been reported.

### SUMMARY OF THE INVENTION

Body weight gain inhibitors having minimal side effects and which can safely be administered over a long period of time and a food composition having inhibitory effect on body weight gain have been desired to be developed.

It has been found that administration or intake of ε-polylysine or a salt thereof provides inhibitory effect on body weight gain and inhibitory effect on fat amount gain even at a low dose and that in a food containing a fat, it provides an action of fat excretion into feces.

That is, the invention provides:

[1] A body weight gain inhibitor containing ε-polylysine or a salt thereof;

[2] The body weight gain inhibitor as described in above-mentioned [1], wherein the body weight gain occurs before reaching obesity;

[3] The body weight gain inhibitor as described in above-mentioned [1], wherein the body weight gain is observed in a patient with obesity;

[4] The body weight gain inhibitor as described in any of above-mentioned [1] to [3], wherein a content of ε-polylysine or a salt thereof is an effective amount for inhibiting body weight gain;

[5] The body weight gain inhibitor as described in above-mentioned [4], wherein the effective amount is from approximately 0.1 mg/kg to approximately 800 mg/kg of body weight per day;

[6] The body weight gain inhibitor as described in any of above-mentioned [1] to [5], wherein a degree of polymerization of ε-polylysine is in the range of from approximately 20 to approximately 40;

[7] A fat amount gain inhibitor containing ε-polylysine or a salt thereof;

[8] A medicament for prevention and/or treatment of obesity containing ε-polylysine or a salt thereof;

[9] A food composition having inhibitory effect on body weight gain inhibiting action containing ε-polylysine or a salt thereof;

[10] The food composition as described in above-mentioned [9], wherein the weight gain occurs before reaching obesity;

[11] The food composition as described in above-mentioned [9], wherein the body weight gain is observed in a patient with obesity;
[12] The food composition as described in any of above-mentioned [9] to [11], wherein a content of ε-polylysine or a salt thereof is an effective amount for inhibiting body weight gain;

[13] The food composition as described in above-mentioned [12], wherein said effective amount is from approximately 0.1 mg/kg to approximately 800 mg/kg of body weight per day;

[14] The food composition as described in any of above-mentioned [9] to [13], wherein a degree of polymerization of ε-polylysine is in the range of from approximately 20 to approximately 40;

[15] The food composition as described in any of above-mentioned [9] to [14], wherein said food composition further contains a fat;

[16] The food composition as described in above-mentioned [15], wherein the content of ε-polylysine is from approximately 0.1% to approximately 5% by weight of said fat contained in the food composition;

[17] The food composition as described in any of above-mentioned [9] to [16], wherein said food composition is a supplement, a functional food, a health food, a food for specified health use or a food for patients;

[18] The food composition as described in any of above-mentioned [9] to [17], wherein said food composition is in the form of a tablet, a pill, a capsule, powders, granules, fine granules, a troche or liquid;

[19] The food composition as described in any of above-mentioned [9] to [18], wherein said food composition is a tablet confectionery, a drop, a candy, a jelly, a beverage, a cookie, a cracker, a biscuit, a chocolate, a margarine or a chewing gum;

[20] The food composition as described in above-mentioned [9], wherein a degree of polymerization of ε-polylysine is in the range of from approximately 20 to approximately 40; a content of ε-polylysine is an amount of from approximately 0.1 mg/kg to approximately 800 mg/kg of body weight per day; said food composition is a supplement, a functional food, a health food, a food for specified health use or a food for patients; and said food composition is in the form of tablet, pill, capsule, powders, granules, fine granules, a troche or liquid;

[21] The food composition as described in above-mentioned [9], wherein a degree of polymerization of ε-polylysine is in a range of from approximately 20 to approximately 40; said food composition further contains a fat; the content of ε-polylysine is from approximately 0.1% to approximately 5% by weight of said fat contained in the food composition; said food composition is a supplement, a functional food, a health food, a food for specified health use or a food for patients; and said food composition is in a form of a tablet, a pill, a capsule, powders, granules, fine granules, a troche or liquid;

[22] A food composition having inhibitory effect on fat amount gain containing ε-polylysine or a salt thereof;

[23] A method for inhibiting body weight gain, which comprises administering to mammals an effective amount of ε-polylysine or a salt thereof;

[24] The method according to above-mentioned [23], wherein said body weight gain occurs before reaching obesity;

[25] The method according to above-mentioned [23], wherein said body weight gain is observed in a patient with obesity;

[26] The method according to above-mentioned [23], wherein said effective amount is from approximately 0.1 mg/kg to approximately 800 mg/kg of body weight per day;

[27] The method according to above-mentioned [23], wherein a degree of polymerization of said ε-polylysine is in the range of from approximately 20 to approximately 40;

[28] A method for inhibiting adipose gain, which comprises administering to mammals an effective amount of ε-polylysine or a salt thereof;

[29] A method for preventing or treating obesity, which comprises administering to mammals an effective amount of ε-polylysine or a salt thereof;

[30] A method for inhibiting body weight gain, which comprises administering to mammals a body weight gain inhibitor comprising ε-polylysine or a salt thereof;

[31] The method according to above-mentioned [30], wherein said body weight gain occurs before reaching obesity;

[32] The method according to above-mentioned [30], wherein said body weight gain is observed in a patient with obesity;

[33] The method according to above-mentioned [30], wherein said inhibitor contains an effective amount of ε-polylysine or a salt thereof for inhibiting body weight gain;

[34] The method according to above-mentioned [33], wherein said effective amount is from approximately 0.1 mg/kg to approximately 800 mg/kg of body weight per day;

[35] The use according to above-mentioned [30], wherein a degree of polymerization of said ε-polylysine is in the range of from approximately 20 to approximately 40;

[36] A method for inhibiting adipose gain, which comprises administering to mammals an adipose gain inhibitor comprising ε-polylysine or a salt thereof;

[37] A method for preventing or treating obesity, which comprises administering to mammals a medicament comprising ε-polylysine or a salt thereof;

[38] A method for inhibiting body weight gain, which comprises administering to mammals a food composition comprising ε-polylysine or a salt thereof;

[39] The method according to above-mentioned [38], wherein said body weight gain occurs before reaching obesity;

[40] The method according to above-mentioned [38], wherein said body weight gain is observed in a patient with obesity;

[41] The method according to above-mentioned [38], wherein said food composition contains an effective amount of ε-polylysine or a salt thereof for inhibiting body weight gain;

[42] The method according to above-mentioned [41], wherein said effective amount is from approximately 0.1 mg/kg to approximately 800 mg/kg of body weight per day;

[43] The method according to above-mentioned [38], wherein a degree of polymerization of said ε-polylysine is in the range of from approximately 20 to approximately 40;

[44] The method according to above-mentioned [38], wherein said food composition further contains a fat;

[45] The method according to above-mentioned [44], wherein the content of the ε-polylysine is from approximately 0.1% to approximately 5% by weight of said fat in the food composition;

[46] The method according to above-mentioned [38], wherein said food composition is a nutritional supplement food, a functional food, a health food, a food for specified health use or a food for patients;

[47] The method according to above-mentioned [38], wherein said food composition is in the form of tablet, pill, capsule, powder, granule, fine granule, troche or liquid;

[48] The method according to above-mentioned [38], wherein said food composition is a tablet, a drop, a candy, a jelly, a drink, a cookie, a cracker, a biscuit, a chocolate, margarine or a chewing gum;

[49] The method according to above-mentioned [38], wherein a degree of polymerization of said ε-polylysine is in the range of from approximately 20 to approximately 40, the content of said ε-polylysine is from approximately 0.1 mg/kg to approximately 800 mg/kg of body weight per day, said food composition is a nutritional supplement food, a functional food, a health food, a food for specified health use or a food for patients, and said food composition is in the form of tablet, pill, capsule, powders, granules, fine granules, troche or liquid;

[50] The method according to above-mentioned [38], wherein a degree of polymerization of said ε-polylysine is in the range of from approximately 20 to approximately 40, said food composition further contains a fat, the content of said ε-polylysine is from approximately 0.1% to approximately 5% by weight of the fat in said food composition, said food composition is a nutritional supplement food, a functional food, a health food, a food for specified health use or a food for patients, and said food composition is in the form of tablet, pill, capsule, powders, granules, fine granules, troche or liquid;

[51] A method for inhibiting adipose gain, which comprises administering to mammals a food composition comprising ε-polylysine or a salt thereof.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1 illustrates a graph showing an action to body weight gain of mice by forced oral administration of EPL in Test Example 1 described later.

FIG. 2 illustrates a graph showing an energy intake of mice in Test Example 1 described later.

FIG. 3 illustrates a graph showing an action to body weight gain of mice by mixed feed administration of EPL in Test Example 2 described later.

FIG. 4 illustrates a graph showing an action to body weight gain of mice by mixed feed administration of EPL in Test Example 4 described later.

FIG. 5 illustrates a graph showing an ingested amount and an energy intake of mice in Test Example 4 described later.

### DETAILED DESCRIPTION OF THE INVENTION

ε-Polylysine (EPL)

The invention provides a body weight gain inhibitor containing ε-polylysine (in the specification, ε-polylysine shall be abbreviated as “EPL”) and a supplement or a food composition having a body weight gain inhibiting action.

First, EPL used in the invention shall be explained. EPL as described herein is a linear polymer in which L-lysine forms an amide bond between a carboxyl group in an α-position and an amino group in an ε-position, and it is represented by Formula (1):

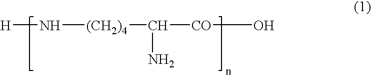

wherein n represents a polymerization degree.

The degree of polymerization (n) is usually in the range of from approximately 2 to approximately 100, preferably from approximately 15 to approximately 50, more preferably from approximately 20 to approximately 40 and particularly preferably from approximately 25 to approximately 35. In particular, EPL of Formula (1) manufactured by Chisso Corporation having a degree of polymerization (n) is in a range of from approximately 25 to approximately 35 which is produced by fermentation using a Streptomyces genus bacterium and which has been used as a food additive over a long period of time is preferred from the viewpoints of safety and food experience. Further, EPL containing approximately 90% by weight or more of EPL having a degree of polymerization (n) of approximately 21 or more and EPL having a degree of polymerization (n) in a range of from approximately 21 to approximately 35 (see, e.g., Japanese Kokai Publication No. 2003-171462) can preferably be used as well. Further, other EPL having a middle polymerization degree (see, e.g., Japanese Kokai Publication No. 2005-006562) can preferably be used as well.

A degree of polymerization of EPL of the invention can be measured by using, for example, a paired ion chromatography (see, e.g., Japanese Kokai Publication No. Hei 9-19288) and a gel penetration chromatography/low angle laser beam scattering light intensity (GPC-LALLS).

EPL used in the invention may form a salt. Salts with acids (for example, inorganic acids and organic acids) and bases (e.g., alkali metals) which are physiologically allowable are used as the above salt of EPL. Among them, acid addition salts which are physiologically allowable are preferred. The salts with inorganic acids include, for example, salts with hydrochloric acid, sulfuric acid, phosphoric acid and hydrobromic acid. The salts with organic acids include, for example, salts with formic acid, acetic acid, trifluoroacetic acid, citric acid, gluconic acid, tartaric acid, lactic acid, phthalic acid, fumaric acid, oxalic acid, maleic acid, succinic acid, malic acid, methanesulfonic acid, p-toluenesulfonic acid, adipic acid, propionic acid, sorbic acid, benzoic acid and ascorbic acid. The salts with the bases include, for example, metal salts, ammonium salts and salts with organic bases. The metal salts include, for example, salts with alkali metals such as sodium and potassium; salts with alkaline earth metals such as calcium, magnesium and barium; and aluminum salts. The salts with organic bases include, for example, salts with trimethylamine, triethylamine, pyridine, picoline, 2,6-lutidine, ethanolamine, diethanolamine, triethanolamine, cyclohexylamine, dicyclohexylamine and N,N′-dibenzylethylenediamine. The salts of EPL can be produced according to conventional methods. Specifically, they can be obtained, for example, by a method in which EPL is mixed with a solution containing a desired acid or base to produce a desired salt and the resulting salt is isolated by filtration or distilling off the solvents.

Hereinafter, EPL and the salts thereof shall be referred altogether to as “EPL”.

EPL of the invention may be obtained by either chemical synthesis or fermentation.

As a process for producing EPL by fermentation, for example, a process culturing a bacterium producing EPL in a liquid culture medium and collecting EPL produced and accumulated in the liquid is known. Such methods include: a method using a Streptomyces albulus subsp. Lysinopolymerus No. 346-D strain (FERM P-3834; hereinafter, referred to as the No. 346-D strain) (see, e.g., Japanese Patent Publication No. Sho 59-20359 and the like), a method using a 11011A-1 strain (FERM BP-1109) which is an S-aminoethyl-L-cysteine resistant variant of the No. 346-D strain (refer to Japanese Patent Publication No. Hei 3-42070 and the like), a method using a 50833 strain (FERM BP-1110) which is a plasmid amplification variant of the No. 346-D strain (see, e.g., Japanese Patent Publication No. Hei 3-42075, Japanese Patent Publication No. Hei 6-75501 and the like), a method using a B21021 strain (FERM BP-5926) which has a tolerance to high concentration of S-aminoethyl-L-cysteine (see, e.g., Japanese Kokai Publication No. Hei 9-173057 and the like), a method using a strain (FERM P-9797) belonging to Streptomyces noursei (see, e.g., Japanese Kokai Publication No. Hei 1-187090 and the like), a method using a Streptomyces sp. SP-72 strain (FERM P-16810) (see, e.g., Japanese Kokai Publication No. 2000-069988 and the like), a method using a Streptomyces sp. SP-66 strain (FERM P-17223) (see, e.g., Japanese Kokai Publication No. 2001-017159 and the like), a method using a Streptomyces herubali color SP-13 strain (FERM P-17845) (see, e.g., Japanese Kokai Publication No. 2002-95466 and the like), a method using a Streptomyces albulus subsp. SP-25 strain (FERM P-17998) (see, e.g., Japanese Kokai Publication No. 2002-95467 and the like) and a method using a Streptomyces lavendurae USE-53 strain (FERM P-18305) (see, e.g., Japanese Kokai Publication No. 2003-52358 and the like).

When EPL is produced by chemical synthesis, it can be produced, for example, by condensing α-amino-protected lysine and then eliminating the protecting group. Publicly known protecting and deprotecting processes for an amino group which can be used for the above production process and a condensing method for amino acids include, for example: processes described in Peptide Synthesis, Interscience Publishers, New York (1966), The Peptide, Academic Press, New York (1965), Peptide Gosei no Kiso to Jikken (Fundamentals and Experiments of Peptide Synthesis), Maruzen (1975), (Course of Biochemical Experiment 1), Chemistry of Protein IV, 205, (1977) and the second series of Pharmaceutical Research and Development Vol. 14, Peptide Synthesis, Hirowaka Publisher.

As a process for producing EPL having a degree of polymerization (n) of from approximately 2 to approximately 19 includes, for example, the process described in Japanese Kokai Publication No. Hei 4-221320 is known. A processes for producing EPL containing approximately 90% by weight or more of EPL having a degree of polymerization (n) of approximately 21 or more and EPL having a degree of polymerization (n) is in a range of from approximately 21 to approximately 35 includes, for example, the processes described in Japanese Kokai Publication No. 2003-171462 are known. Further, EPL used in the invention can be produced as well by a process described in Japanese Kokai Publication No. 2005-006562.

EPL having a smaller degree of polymerization (n) can be produced as well by hydrolyzing EPL obtained by the above processes with protease (see, e.g., Japanese Kokai Publication No. Hei 4-221320 and the like).

Pharmaceutical and Food Composition Containing EPL and a Salt Thereof

EPL and the salts thereof have a body weight gain inhibiting activity and a fat amount gain inhibiting activity, and therefore EPL and the salts thereof can be used to treat animals as, for example, a body weight gain inhibitor, a fat amount gain inhibitor and a medicament for prevention and/or treatment of obesity (hereinafter abbreviated as “the pharmaceuticals of the invention”) and as, for example, a food composition having inhibitory effect on body weight gain and a food composition having inhibitory effect on fat amount gain (hereinafter abbreviated as “the food compositions of the invention”).

EPL prevents, as described above, absorption of fat by preventing conversion of an emulsion formed by an ingested fat into a micelle, and therefore the food composition is particularly preferably used for foods containing larger fat ingredients.

In respect to a preferred method for administering the pharmaceuticals or ingesting the food compositions, administration or ingestion after a meal is particularly preferred judging from a fat absorption preventing mechanism of EPL. However, it may be a punctual administrating or ingesting form as is the case with Test Example 1 described later. In the case of the food compositions of the invention, an administrating or ingesting method shall not specifically be restricted.

The animals which are objects for application are preferably vertebrates such as human beings, dogs, cats, poll parrots, myna birds, parrots, guinea pig, rats, mice, pigs, sheep, cattle, monkeys, frogs, salamanders, goldfishes, carps and crucian carps. They are more preferably mammals, particularly preferably human beings. The animals which are objects for application may be any ones as long as they are animals which is intended to avoid body weight gain or fat amount gain or animals which is intended to prevent and/or treat obesity. They may be animals having a hereditary risk of body weight gain or may be animals suffering from lifestyle diseases such as diabetes, hypertension and/or hyperlipemia.

Body weight gain may be body weight gain occurs before reaching obesity and may be observed in a patient with obesity. According to the standard of the WHO, obesity is defined by a BMI (body mass index: body weight (kg)/[body height (m)]²) of 30 or more, and in the case of the Japanese, obesity means that the BMI is 25 or more (according to the standard of Japan Society for the Study of Obesity). The same shall apply in the invention.

EPL and the salts thereof can be used, as described above, as a fat amount gain inhibitor or a food composition having a fat amount gain inhibiting action. Among them, they can be used preferably as a neutral fat amount gain inhibitor or a food composition having an action for inhibiting a neutral fat amount gain, and in particular, they can be used preferably as a triglyceride amount gain inhibitor or a food composition having an action for inhibiting a triglyceride amount gain.

Tissues or organs which are inhibited from increasing fat amount by the fat amount gain inhibitor or the food composition having a fat amount gain inhibiting action according to the invention include, for example, tissues or organs containing a lot of adipose cells such as liver, epididymis fat tissue and lateral region fat tissue. Further, the fat amount gain inhibitor or the food composition having inhibitory effect on fat amount gain according to the invention have an action to inhibit increase of lipid content in blood.

Further, EPL and the salts thereof have an action to inhibit increase of total cholesterol in blood. More specifically, EPL and the salts thereof have an action to inhibit increase of cholesterol other than HDL without affecting HDL cholesterol in blood. Accordingly, EPL and the salts thereof can be used as a cholesterol increase inhibitor or a food composition having a cholesterol increase inhibiting action. Preferably, EPL and the salts thereof can be used as a cholesterol increase inhibitor which inhibits increase of cholesterol other than HDL without inhibiting HDL cholesterol or a food composition having inhibitory effect on increase of cholesterol other than HDL without inhibiting HDL cholesterol.

Further, EPL and the salts thereof have an action to promote excretion of fat into feces. Accordingly, EPL and the salts thereof can be used as an agent for fat excretion into feces or a food composition having an action to promote fat excretion into feces.

Pharmaceutical of the Invention

When EPL is used as the pharmaceutical described above, it can be used by a publicly known method. Specifically, it can be used as described below.

The pharmaceutical of the invention is preferably used orally in the forms of a tablet provided with a sugarcoating or coating if necessary, a pill, a capsule (including a soft capsule, a hard capsule and a microcapsule), a powder, a granule, a fine granule, a troche and a liquid drug (including a syrup, an emulsion and a suspension).

The pharmaceutical (pharmaceutical composition) of the invention can be blended with a physiologically acceptable carrier as long as it is not disturbing the effects of the invention. Various organic or inorganic carrier substances which are conventionally used as formulation materials are used as the physiologically acceptable carrier. They include fillers, binders, disintegrating agents and lubricants in the solid preparation; and solvents, dissolution auxiliary agents, suspending agents, buffering agents, thickeners and emulsifying agents in the liquid preparation. Also, formulation additives such as colorants, sweetening agents and antioxidants can be used as well if necessary. Further, the preparation of the invention may be coated.

The fillers include, for example, lactose, white sugar, D-mannitol, D-sorbitol, starch, a-starch, dextrin, crystalline cellulose (for example, fine crystalline cellulose and the like), low substituted hydroxypropyl cellulose, carboxymethylcellulose sodium, gum arabic, dextrin, pullulan, light anhydrous silicic acid, synthetic aluminum silicate and magnesium aluminometasilicate. The binders include, for example, α-starch, sucrose, gelatin, macrogol, gum arabic, methyl cellulose, carboxymethyl cellulose, carboxymethyl cellulose sodium, crystalline cellulose, white sugar, D-mannitol, trehalose, dextrin, pullulan, hydroxypropyl cellulose (HPC), hydroxypropyl methyl cellulose (HPMC) and polyvinylpyrrolidone (PVP). The disintegrating agents include, for example, lactose, white sugar, starch, carboxymethyl cellulose, carboxymethyl cellulose calcium, cross-linked polyvinylpyrrolidone, carmellose sodium, cross carmellose sodium, carboxymethyl starch sodium, light anhydrous silicic acid, low substituted hydroxypropyl cellulose, cation exchange resins, partial α-starch and corn starch. The lubricants include, for example, stearic acid, magnesium stearate, calcium stearate, talc, waxes, colloidal silica, DL-leucine, sodium laurylsulfate, magnesium laurylsulfate, macrogol and aerosil.

The solvents include, for example, water for injection, saline, Ringer's solutions, alcohols, propylene glycol, polyethylene glycol, medium chain fatty acid triglycerides (MCT) and vegetable oils (for example, safflower oil, sesame oil, corn oil, olive oil, cotton seed oil and soybean lecithin). The dissolution auxiliary agents include, for example, polyethylene glycol, propylene glycol, D-mannitol, trehalose, benzyl benzoate, ethanol, trisaminomethane, cholesterol, triethanolamine, sodium carbonate, sodium citrate, sodium salicylate and sodium acetate. The suspending agents include, for example, surfactants such as stearyltriethanolamine, sodium laurylsulfate, laurylaminopropionate, lecithin, benzalkonium chloride, benzethonium chloride and monostearic acid glycerin; hydrophilic polymers such as, for example, polyvinyl alcohol, polyvinylpyrrolidone, carboxymethyl cellulose sodium, methyl cellulose, hydroxymethyl cellulose, hydroxyethyl cellulose and hydroxypropyl cellulose; polysorbates and polyoxyethylene-hardened castor oil. The buffering agents include, for example, buffer solutions of phosphates, acetates, carbonates and citrates. The thickeners include, for example, natural gums, cellulose derivatives and the like. The emulsifying agents include, for example, fatty acid esters (for example, sucrose fatty acid esters, glycerin fatty acid esters, sorbitan fatty acid esters and propylene glycol fatty acid esters), waxes (for example, yellow beeswax, rapeseed hydrogenated oils, safflower hydrogenated oils, palm hydrogenated oils, sitosterol, stigmasterol, campesterol, brasicasterol, cacao fat powder, carnauba wax, rice wax, Japan tallow and paraffin) and lecithin (for example, egg-yolk lecithin and soybean lecithin).

The colorants include, for example, water-soluble food tar coloring matters (e.g., food coloring matters such as food red No. 2 and No. 3, food yellow No. 4 and No. 5 and food blue No. 1 and No. 2), water-soluble lake coloring matters (for example, aluminum salts of the water-soluble food tar coloring matters described above and the like) and natural coloring matters (for example, β-carotene, chlorophyll and red iron oxide). The sweetening agents include, for example, sucrose, lactose, saccharin sodium, dipotassium glycyrrhizinate, aspartame and stevia. The antioxidants include, for example, sulfites, ascorbic acid and alkaline metal salts and alkaline earth metal salts thereof.

In respect to a tablet, granules and fine granules, coatings may be provided by an ordinary method using coating base materials for the purposes of masking of taste, improving light stability, improving appearance and enteric property. The above coating base materials include sugarcoating base materials, water-soluble film coating base materials and enteric film coating base materials.

The sugarcoating base materials include, for example, sucrose, and at least one of talc, precipitated calcium carbonate, gelatin, gum arabic, pullulan and carnauba wax may be used in combination therewith.

The water-soluble film coating base materials include, for example, cellulose polymers such as hydroxypropyl cellulose (HPC), hydroxypropyl methyl cellulose (HPMC), ethyl cellulose, hydroxyethyl cellulose and methyl hydroxyethyl cellulose; synthetic polymers such as polyvinylacetal diethylaminoacetate, aminoalkyl methacrylate copolymer E (Eudragit E®, Rohm Pharma Co., Ltd.) and polyvinylpyrrolidone; and polysaccharides such as pullulan and the like. The enteric film coating base materials include, for example, cellulose polymers such as hydroxypropyl methyl cellulose phthalate, hydroxypropyl methyl cellulose acetate succinate, carboxymethyl ethyl cellulose and cellulose acetate phthalate; acrylic acid polymers such as methacrylic acid copolymer L (Eudragit L®, Rohm Pharma Co., Ltd.), methacrylic acid copolymer LD (Eudragit L-30D55®, Rohm Pharma Co., Ltd.) and methacrylic acid copolymer S (Eudragit S®, Rohm Pharma Co., Ltd.); and natural substances such as shellac and the like. The above coating base materials may be coated alone, or two or more kinds thereof may be mixed in a suitable proportion and then coated, and two or more kinds thereof may be coated in order.

A content of EPL in the pharmaceutical (formulation) of the invention is in an optional range of usually approximately 0.001% to approximately 99% by weight, preferably approximately 0.01% to approximately 80% by weight and more preferably approximately 0.1% to approximately 50% by weight based on the whole part of the pharmaceutical.

A dose of EPL or the salt thereof used in the pharmaceutical of the invention may be in a range of an effective amount of EPL or the salt thereof to inhibit body weight gain or fat amount gain. For example, when it is administered to an adult for the purpose of inhibiting body weight gain, a dose of EPL is, though varied depending on an administered objective, an administering manner and an ingested amount, usually approximately 0.1 mg/kg to approximately 800 mg/kg of body weight per day, preferably approximately 0.1 mg/kg to approximately 400 mg/kg of body weight per day, more preferably approximately 0.1 mg/kg to approximately 15 mg/kg of body weight per day, particularly preferably approximately 0.5 mg/kg to approximately 10 mg/kg of body weight per day and most preferably approximately 0.5 mg/kg to approximately 5 mg/kg of body weight per day.

If a dose of EPL is too small, the effects are not exhibited, and if it is too large, absorption of other nutritional elements such as fat-soluble vitamins is likely to be disturbed. The respective doses given above as the examples are preferred from the viewpoint that the effects are exhibited without affecting food faddiness and the amount of food intake. In the case of other animals, the same amount can be administered.

Further, EPL or the salt thereof used in the pharmaceutical of the invention can be used in combination with diabetes therapeutic agents, diabetic complication therapeutic agents, anti-hyperlipemia agents, hypotensive agents and diuretic agents (hereinafter abbreviated as the combination drugs). In the specification, “use in combination” may be either a form of administering them as separate formulations or a form of combination drug as a single formulation.

When they are used in combination as the separate formulations, the administration timings of EPL or the salt thereof used in the pharmaceutical of the invention and the combination drugs shall not be restricted, and they may be administered simultaneously or may be administered at any administration interval. Further, two or more kinds of the combination drugs may be used in combination in a suitable proportion.

Food Composition of the Invention

When EPL is used for the food compositions described above, it can be used by an ordinary method. Specifically, it can be used, for example, in a manner described below.

The food compositions of the invention may be any one as long as it contains EPL and can be ingested orally by animals. The food compositions shall not be restricted to specific kinds or forms. The food compositions include, for example, sweet stuffs such as drops, candies and chewing gums; western confectioneries such as cookies, crackers, biscuits, potato chips, breads, cakes, chocolates, doughnuts, pudding and jelly; Japanese confectioneries such as rice crackers, sweet jelly of beans, rice cakes stuffed with bean jam, rice dumplings covered with bean jam, buns with bean-jam filling and sponge cakes; frozen deserts such as ice creams, ice candies, sherbets and gelatos; breads such as breads, French rolls and crescent rolls; noodles such as Japanese wheat noodles, buckwheat noodles and kishimen noodles; fish cakes such as steamed fish pastes and fish meat sausages; meat products such as hams, sausages, hamburger steaks and corned beef; seasonings such as salts, peppers, soybean pastes, soy sauces, sauces, dressings, mayonnaises, tomato sauces, sweetenings and spices; foods cooked on a hot plate such as akashiyaki, octopus balls, monnjayaki, savory pancakes, fried Chinese noodles and fried wheat noodles; dairy products such as cheeses and hard type yogurts; various prepared foods such as fermented soybeans, deep-fried tofu, tofu, devil's tongues, rice dumplings, pickles, fishes boiled in soy sauce, potstickers, su my, croquettes, sandwiches, pizzas, hamburgers and salads; livestock food products such as beef, pork and chicken; aquatic products such as shrimps, scallop, freshwater clam and sea tangle; various powders obtained by powdering vegetables, fruits, plants, yeasts and algae; materials obtained by powdering and solidifying oils & fats and fragrances (vanilla, citrus and bonito, etc.); and beverages.

The beverages include foods and drinks such as soup and miso soup; powder foods and drinks such as instant coffee, instant tea, instant milk, instant soup and instant miso soup; alcoholic beverages such as whisky, bourbon, spirits, liqueur, wine, fruit wine, rice wine, Chinese wine, distilled spirit, beer, non-alcoholic beer having an alcohol content of 1% or less, sparkling liquor and distilled spirit and aerated water; and non-alcoholic beverages such as beverages containing fruit juices (for example, juices of apple, orange, grape, banana, pear and Japanese apricot), beverages containing vegetable juices (for example, vegetable juices of tomato, carrot, celery, cucumber and water melon), beverages containing vegetable juices and fruit juices, soft drinks, cow milk, soy milk, milk beverages, yogurts of a drink type, coffee, cocoa, tea beverages (tea, green tea, barley tea, brown rice tea, natural leaf tea, refined green tea, roasted green tea, oolong tea, black tea, Rooibos tea, rose tea, mum tea and herb tea (for example, mint tea and jasmine tea)), nutrition-supplement drinks, sport beverages and mineral water.

The preferred examples of the above food composition include, for example, jellies, beverages, tablet confectioneries, drops, candies, cookies, crackers, biscuits, chocolates, margarines or gum.

EPL used in the food composition of the invention has an action of excreting fat contained in foods into feces and can inhibit body weight gain and fat gain. Thus, when fat is contained in the food composition of the invention, the fat can be excreted more efficiently into feces to make it possible to inhibit more efficiently body weight gain and fat gain. Accordingly, a food composition containing fat can suitably used as well in the invention. Such food composition containing fat includes, for example, cookies, crackers, biscuits, potato chips, breads, cakes, chocolates, doughnuts, pudding, sponge cakes, ice creams, hams, sausages, hamburgs, corned beef, dressings, mayonnaises, cheeses, yogurts, potstickers, su my, croquettes, pizzas, hamburger steaks, beef, pork, chicken, instant milk, cow milk, soy milk, milk beverage, soup and instant soup.

The food composition of the invention may be prepared in the forms of a functional food, a health food, a food for specified health use, a food for patients and a nutritional supplement food, and it is preferably prepared in the form of a functional food. The shape of the food composition of the invention includes tablet form, pill form, capsule (including a hard capsule, a soft capsule and a microcapsule) form, powder form, granular form, fine granular form, troche form and liquid form (including a syrup form, an emulsion form and a suspension form), tablet form and capsule form are preferred.

Among the food compositions of the invention, preferred are tablet form and capsule form, and particularly preferred are a functional food having tablet form and a functional food having capsule form.

In the specification, a supplement not only means a nutritional supplement food and a dietary functional food for supplementing nutrients, but also means a health supplement and a health functional food having functions (for example, body weight gain inhibition and fat amount gain inhibition) useful for maintaining, recovering and enhancing health.

The food composition of the invention can be produced, for example, by adding EPL to a food by a publicly known method. Specifically, for example, the tablet-shaped food composition can be produced by adding EPL to raw materials such as a filler (for example, lactose, white sugar, mannitol and the like), a sweetening agent and a colorant, mixing them and molding the mixture into tablets by applying pressure using a tableting machine. Other materials (for example, vitamins such as vitamin C, minerals such as iron, a vegetable fiber and the like) can be added as well if necessary. The capsule-shaped food composition can be produced, for example, by filling a capsule with the liquid, suspended, paste-like, powder-like or granular food composition containing EPL or covering it with a capsule base material and molding.

The food composition of the invention can be blended with a physiologically acceptable filler and the like in addition to food materials, food additives, various nutrients, vitamins, flavoring stuffs (for example, cheese and chocolate) and the like which are usually used as long as the effects of the invention are not disturbed. Various organic or inorganic carrier substances which are conventionally used are used as the physiologically acceptable carrier, and they include fillers, binders, disintegrating agents, lubricants, colorants, sweetening agents, preservatives, antioxidants, thickeners, emulsifying agents and the like. The food additives include colorants, sweetening agents, preservatives, antioxidants, fragrance-providing agents and the like. Further, other materials, for example, minerals such as iron and food fibers such as pectin, carrageenan and mannan may be contained.

The fillers, the binders, the disintegrating agents, the lubricants, the solvents, the dissolution auxiliary agents, the suspending agents, the buffering agents, the thickeners, the colorants, the sweetening agents, the preservatives and the antioxidants include the same materials as those used for the pharmaceutical of the invention.

The vitamins may be water-soluble or fat-soluble and include, for example, retinol palmitate, tocopherol, bisbentiamine, riboflavin, pyridoxine hydrochloride, cyanocobalamine, sodium ascorbate, cholecalciferol, nicotinamide, calcium pantothenate, folic acid, biotin and choline bitatrate.

The tablet-shaped, granular and fine granular-shaped food compositions may be coated with a coating material by a publicly known method for the purposes of masking of taste, improving light stability, improving appearance and enteric property. The above coating base material includes the same materials as those used for the pharmaceutical of the invention, and it can be coated in the same manner.

A content of EPL in the food composition of the invention is usually from approximately 0.001% to approximately 10% by weight, preferably from approximately 0.01% to approximately 5% by weight and more preferably about from approximately 0.05% to approximately 1% by weight based on the whole part of the food composition. If a content of EPL is too small, the effects are not exhibited, and if it is too large, absorption of other nutrient components such as fat-soluble vitamins is likely to be disturbed.

When the food composition contains fat, a content of EPL based on the fat contained in the food composition of the invention is in a range of usually from approximately 0.05% to approximately 5% by weight, preferably from approximately 0.1% to approximately 5% by weight and more preferably from approximately 0.2% to approximately 2.5% by weight. If a content of EPL is too small, the effects are not exhibited, and if it is too large, absorption of other nutrient components such as fat-soluble vitamins is likely to be disturbed.

The food composition thus obtained is safe and therefore can be given to, for example, vertebrates.

An amount of the food composition of the invention to be ingested may be in a range of an effective amount of EPL or the salt thereof in which body weight gain or fat amount gain is inhibited. For example, when the food composition of the invention is ingested by an adult for the purpose of inhibiting body weight gain, an amount of EPL to be ingested is, though varied depending on an objective of ingesting it, an ingesting manner and an amount of food intake, usually from approximately 0.1 mg/kg to approximately 800 mg/kg of body weight per day, preferably from approximately 0.1 mg/kg to approximately 400 mg/kg of body weight per day, more preferably from approximately 1 mg/kg to approximately 400 mg/kg of body weight per day and particularly preferably from approximately 40 mg/kg to approximately 400 mg/kg of body weight per day.

If a dose of EPL is too small, the effects are not exhibited, and if it is too large, absorption of other nutritional elements such as fat-soluble vitamins is likely to be disturbed. The respective doses described above are preferred from the viewpoint that the effects are exhibited without affecting food faddiness and food intake. In the case of other animals, the same amount can be administered.

### SPECIFIC EXAMPLES

The following examples are for illustrative purposes only and are not intended, nor should they be interpreted to, limit the scope of the invention.

### Test Example 1

Test example 1 illustrates the effect on body weight gain and fat amount gain of mice ingesting fatty food by forced oral administration of EPL.

Experimental method: C57BL/6 mice (male, 4 weeks old, CLEA Japan, Inc.) were pre-bred for a week, and then they were bred for 16 weeks under ad libitum feeding of diets having blend composition shown in Table 1 and water for each group. AIN-93G (mineral mixture) and AIN-93 (vitamin mixture) manufactured by Oriental Yeast Co., Ltd. were used for a mineral mixture and a vitamin mixture, respectively. In an EPL-administered group, 0.5 mL (a concentration was controlled respectively so that a dose of EPL was 1 mg or 10 mg per kg body weight) of an EPL aqueous solution prepared by dissolving EPL (degree of polymerization (n)=25 to 35, number average molecular weight (Mn)=4,090, weight average molecular weight (Mw)=4,700, molecular weight distribution (Mw/Mn)=1.14, Chisso Corporation) in purified water was orally administered once a day in the morning using a sonde. All animals were bred at a temperature of 24° C. and a humidity of 60% under the 12 hour light-dark cycle (light period: 8:00 AM to 8:00 PM).

The ingested amounts and the body weights were measured every week until the sixteenth week. After the measurement in the sixteenth week, the mice were killed, and liver weight and fat content of the liver were measured respectively. After extracting the fat according to a Bligh and Dyer method (Bligh E. G. and W. E. Dyer Can. J. Biochem. Physiol. 31, 911 (1959)), a fat content of the liver was measured by means of a measuring kit (triglyceride E-Test Wako, Wako Pure Chemical Industries, Ltd.). Specifically, 1 mL of purified water was added to 200 mg of the liver tissue to homogenize the mixture, and 1.25 mL of purified water and 5.0 mL of a methanol-chloroform mixed solution (1:1 V/V) were added and mixed for one minute using a vortex mixer. Then, it was separated by centrifugation at 1000 g for 10 minutes, and the chloroform layer was concentrated and dried up, followed by measuring the fat content using the measuring kit described above.

The standard group is composed of 6 mice, and the control group, the EPL 1 mg/kg-administered group and the EPL 10 mg/kg-administered group are composed of 7 mice per group.

TABLE 1

| | | | Control Group | |
|---|---|---|---|---|
| | Standard Group | | EPL-Administered Groups | |
| Diet Composition | (g) | (kcal) | (g) | (kcal) |
| Milk butter | 6 | 50.4 | 45 | 378 |
| Corn starch | 36.5 | 146 | 17.5 | 70 |
| Sucrose | 10 | 40 | 10 | 40 |
| Casein | 20 | 80 | 20 | 80 |
| Cellulose | 3 | 0 | 3 | 0 |
| Mineral mixture | 3.5 | 0 | 3.5 | 0 |
| Vitamin mixture | 1 | 3.9 | 1 | 3.9 |
| Water | 20 | 0 | 0 | 0 |
| Total | 100 | 320 | 100 | 572 |

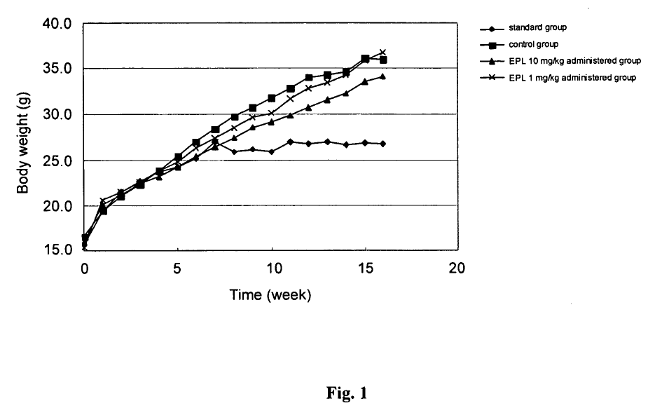

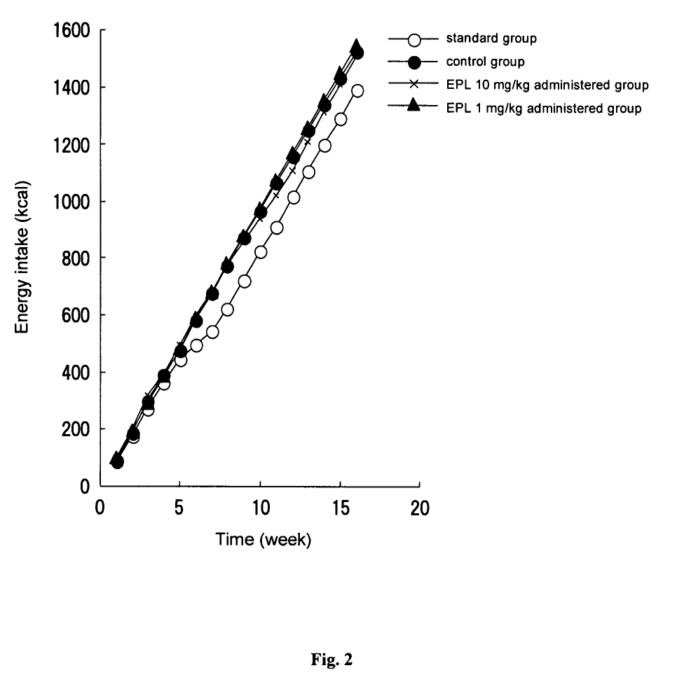

Results: Both of the EPL 1 mg/kg-administered group and the EPL 10 mg/kg-administered group were inhibited in body weight gain as compared with the control group. It was thus shown that EPL inhibited body weight gain dose dependently (FIG. 1). It was shown that a triglyceride content of the liver was small in both of the EPL 1 mg/kg-administered group and the EPL 10 mg/kg-administered group as compared with the control group and that EPL inhibited increase in the triglyceride content dose dependently (Table 2). No difference in energy intake calculated by a conventional food calorie table was observed between the standard group, the control group, the EPL 1 mg/kg-administered group and the EPL 10 mg/kg-administered group (FIG. 2).

TABLE 2

| | | | EPL 1 mg/kg- | EPL 10 mg/kg- |
|---|---|---|---|---|
| | Standard | Control | Administered | Administered |
| | Group | Group | Group | Group |
| Liver weight | 1.28 | 1.59 | 1.57 | 1.35 |
| (g) | | | | |
| Triglyceride | 21.05 | 85.64 | 71.94 | 61.00 |
| content | | | | |
| (mg/g liver) | | | | |

### Test Example 2

Test example 2 illustrates the effect on body weight gain of mice by administration of a mixed diet of EPL

Experimental method: C57BL/6 mice (male, 4 weeks old, CLEA Japan, Inc.) were pre-bred for a week, and then they were bred for 6 weeks under ad libitum feeding of diets having blend compositions shown in Table 3 and water for each group (n=10). The same EPL as in Test Example 1 was used. AIN-93G (mineral mixture) and AIN-93 (vitamin mixture) manufactured by Oriental Yeast Co., Ltd. were used for a mineral mixture and a vitamin mixture, respectively. All animals were bred at a temperature of 24° C. and a humidity of 60% under the 12 hour light-dark cycle (light period: 8:00 AM to 8:00 PM), and the body weights were measured every week until the sixth week.

TABLE 3

| Diet Composition | | EPL-Administered |
|---|---|---|
| (% by weight) | Control Group | Group |
| Milk butter | 45 | 45 |
| Corn starch | 17.5 | 16.5 |
| Sucrose | 10 | 10 |
| Casein | 20 | 20 |
| Cellulose | 3 | 3 |
| Mineral mixture | 3.5 | 3.5 |
| Vitamin mixture | 1 | 1 |
| EPL | 0 | 1 |
| Total | 100 | 100 |

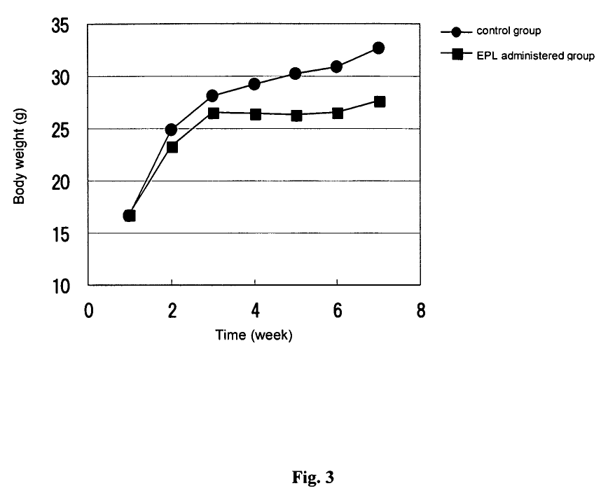

Results: It was shown that the EPL-administered group was inhibited in body weight gain as compared with the control group and that EPL inhibited body weight gain (FIG. 3).

### Test Example 3

Test example 3 illustrates the action on fat excretion into feces in mice ingesting a fatty food by forced oral administration of EPL

Experimental method: The feces of the respective groups of a control group (7 mice/group), an EPL 1 mg/kg-administered group (7 mice/group) and an EPL 10 mg/kg-administered group (7 mice/group) in the fourteenth week of fatty food-ingesting C57BL/6 mice which were bred in the same manner as in Test Example 1 were obtained and freeze-dried. After extracting the fat according to the Bligh and Dyer method, the feces triglyceride contents were measured using the measuring kit (triglyceride E-Test Wako, Wako Pure Chemical Industries, Ltd.). Specifically, 6.75 mL of purified water was added to 1.0 g of the freeze-dried feces to homogenize the mixture, and 15.0 mL of a methanol-chloroform mixed solution (1:1 V/V) was added and shaken for 30 minutes. Then, it was separated by centrifugation at 1000 g for 10 minutes, and the chloroform layer was concentrated and dried up, followed by measuring the fat content using the measuring kit described above.

Results: It was shown that a triglyceride (TG) content in feces in the EPL-administered groups grew larger depending on an administration concentration of EPL as compared with the control group of no-EPL-administration (Table 4).

TABLE 4

| | TG mg/g of Dry Feces |
|---|---|
| Control Group | 10.46 |
| (no-EPL-administered group) | |
| EPL 10 mg/kg-administered group | 11.97 |
| EPL 1 mg/kg-administered group | 10.79 |

### Test Example 4

Test example 4 illustrates the effect on mice by administration of a mixed diet of EPL.

Experimental method: C57BL/6 mice (male, 8 weeks old, CLEA Japan, Inc.) were pre-bred for a week, and then they were bred (5 mice/cage) for 60 days under ad libitum feeding of diets having blend compositions shown in Table 5 and water for each group (10 mice/group). The same EPL as in Test Example 1 was used. AIN-93G (mineral mixture) and AIN-93 (vitamin mixture) manufactured by Oriental Yeast Co., Ltd. were used for a mineral mixture and a vitamin mixture, respectively. All animals were bred at a temperature of 24° C. and a humidity of 60% under the 12 hour light-dark cycle (light period: 8:00 AM to 8:00 PM), and the body weights and the ingestion amounts were measured every week until the sixtieth day.

TABLE 5

| Diet | | | | | |
|---|---|---|---|---|---|
| Composition | Standard | Control | 0.1% EPL | 0.2% EPL | 0.4% EPL |
| (weight ratio) | Group | Group | Group | Group | Group |
| Milk Butter | 6 | 45 | 45 | 45 | 45 |
| Corn Starch | 56 | 17 | 17 | 17 | 17 |
| Sucrose | 10 | 10 | 10 | 10 | 10 |
| Casein | 20 | 20 | 19.9 | 19.8 | 19.6 |
| Cellulose | 3 | 3 | 3 | 3 | 3 |
| Powder | | | | | |
| Mineral | 3.6 | 3.6 | 3.6 | 3.6 | 3.6 |
| Mixture | | | | | |
| Choline | 0.4 | 0.4 | 0.4 | 0.4 | 0.4 |
| Hydrochloride | | | | | |
| Vitamin | 1 | 1 | 1 | 1 | 1 |
| Mixture | | | | | |
| Water | 50 | 0 | 0 | 0 | 0 |
| EPL | 0 | 0 | 0.1 | 0.2 | 0.4 |
| Total | 150 | 100 | 100 | 100 | 100 |

The feces were obtained by group in the seventeenth day and the thirty fifth day and freeze-dried, and then the weights, the fat contents in feces and the cholesterol contents in feces were measured. The fat content in feces was measured in the same manner as in Test Example 3. The total fat was extracted from the feces by a Bligh and Dyer method (Can. J. Biochem. Physiol. 31: 911 (1959)), and the cholesterol content in feces was measured using Cholesterol E-Test Wako of Wako Pure Chemical Industries, Ltd.

Further, during the morning in 60th day and 61st day, the mouse was anesthetized, and then heparin blood drawing, the liver and the fat tissue were obtained and weight were measured. A fat content in the liver was measured in the same manner as in Test Example 1. A cholesterol content in the serum was measured in the same manner as in the measurement (from the feces) described above.

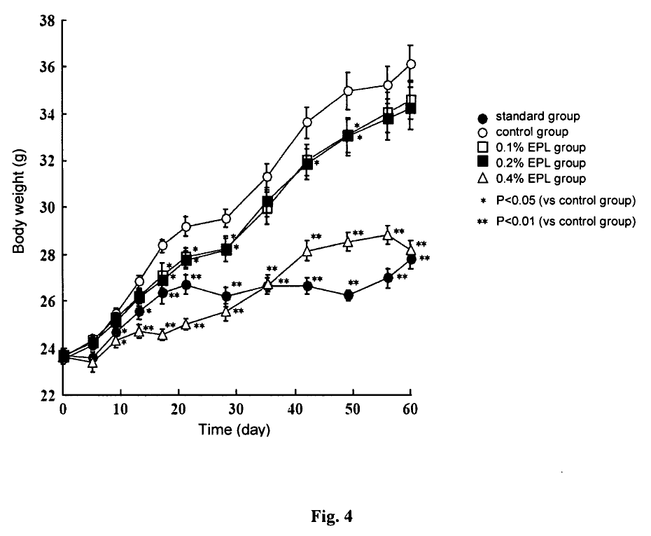

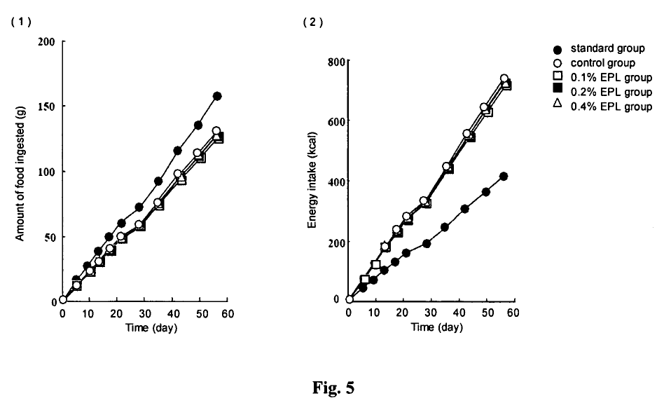

Results: All of the 0.1% EPL group, the 0.2% EPL group and the 0.4% EPL group were inhibited in body weight gain as compared with the control group. It was thus shown that EPL inhibited body weight gain dose dependently (FIG. 4, Table 6). Further, a difference in the liver weight was not observed between the control group and the 0.1% EPL group, but it was shown that the liver weight was smaller in both of the 0.2% EPL group and the 0.4% EPL group than in the control group and that EPL inhibited increase in the liver weight dose dependently (Table 6, showing the average values of the respective groups). Similarly, it was shown as well that EPL inhibited increase in triglyceride content of the liver. Further, it was shown that the epididymis fat tissue weight and the side abdominal fat tissue weight were smaller in all of the 0.1% EPL group, the 0.2% EPL group and the 0.4% EPL group than in the control group and that EPL inhibited increase in the fat tissue weight dose dependently (Table 6, showing the average values of the respective groups). No difference in the ingestion amount and the energy intake calculated by a conventional food calorie table was observed between the control group, the 0.1% EPL group, the 0.2% EPL group and the 0.4% EPL group. The standard group had a larger amount of food ingested but had a smaller energy intake as compared with the other groups (FIG. 5, Table 6). The amounts of EPL ingested of the respective groups are shown in Table 6. The amount of food ingested was measured until 56th day. The amount of food ingested of each group was changed scarcely through a breeding period.

TABLE 6

| | Standard | Control | 0.1% EPL | 0.2% EPL | 0.4% EPL |
|---|---|---|---|---|---|
| | Group | Group | Group | Group | Group |
| Weight (0 day) (g) | 23.68 | 23.56 | 23.65 | 23.71 | 23.65 |
| Weight (60th day) (g) | 27.79 | 36.13 | 34.81 | 34.23 | 28.18 |
| Liver weight (g) | 1.29 | 1.47 | 1.47 | 1.36 | 1.28 |
| Liver Triglyceride Content (g/liver) | 15.80 | 44.17 | 51.81 | 40.75 | 28.35 |
| Epididymis Fat Tissue Weight (60th day) (g) | 0.42 | 1.85 | 1.44 | 1.38 | 0.71 |
| Side Abdominal Fat Tissue Weight (60th day) (g) | 0.10 | 0.54 | 0.40 | 0.37 | 0.19 |
| Ingestion Amount (g/day/mouse) | 2.93 | 2.43 | 2.32 | 2.38 | 2.40 |
| Energy intake (kcal/day/mouse) | 7.78 | 13.83 | 13.24 | 13.54 | 13.67 |
| EPL intake (mg/day/mouse) | 0 | 0 | 2.32 | 4.75 | 9.52 |
| EPL intake (mg/kg/day) | 0 | 0 | 81.5 | 166.7 | 370.4 |

Next, the blood triglyceride concentration was smaller in all of the 0.1% EPL group, the 0.2% EPL group and the 0.4% EPL group than in the control group. It was thus shown that EPL inhibited blood triglyceride dose dependently (Table 7, showing the average values of the respective groups). The blood total cholesterol concentration was smaller in all of the 0.1% EPL group, the 0.2% EPL group and the 0.4% EPL group than in the control group. It was thus shown that EPL inhibited blood total cholesterol dose dependently. However, no difference in the blood HDL cholesterol concentration was observed between the control group and the respective EPL groups. From the observation above, it became apparent that EPL inhibited cholesterols (non-HDL cholesterols) other than HDL without inhibiting HDL cholesterol in the blood (Table 7, showing the average values of the respective groups).

TABLE 7

| | Standard | Control | 0.1% EPL | 0.2% EPL | 0.4% EPL |
|---|---|---|---|---|---|
| | Group | Group | Group | Group | Group |
| Blood Triglyceride | 67.0 | 129.2 | 118.8 | 106.3 | 93.1 |
| Concentration (mg/dl) | | | | | |
| Blood Total Cholesterol | 117.0 | 187.8 | 167.6 | 170.7 | 165.0 |
| Concentration (mg/dl) | | | | | |
| Blood HDL Cholesterol | 43.0 | 67.1 | 72.8 | 64.7 | 66.3 |
| Concentration (mg/dl) | | | | | |
| Blood Non-HDL Cholesterol | 74.0 | 120.7 | 94.8 | 102.1 | 98.7 |
| Concentration (mg/dl) | | | | | |

Further, it was shown that the triglyceride amount and the cholesterol amount in the feces were increased in the EPL-administered groups depending on administered concentration of EPL (Table 8). The contents and the amounts of triglyceride and cholesterol in the feces were measured respectively in the 17th day and the 35th day and shown by the average values.

TABLE 8

| | Standard | Control | 0.1% EPL | 0.2% EPL | 0.4% EPL |
|---|---|---|---|---|---|
| | Group | Group | Group | Group | Group |
| Triglyceride Content in Feces | 3.38 | 4.38 | 7.23 | 8.77 | 11.08 |
| (mg/g of dried feces) | | | | | |
| Triglyceride Amount in Feces | 0.62 | 0.50 | 0.99 | 1.80 | 2.70 |
| (mg/day/mouse) | | | | | |
| Cholesterol Content in Feces | 3.85 | 6.35 | 11.01 | 14.26 | 14.41 |
| (mg/g of dried feces) | | | | | |
| Cholesterol Amount in Feces | 0.72 | 0.77 | 1.61 | 3.05 | 3.78 |
| (mg/day/mouse) | | | | | |

### Example 1

### Tablet-Shaped Functional Food

Fine crystalline cellulose and mannitol are suitably added to 60 g of EPL while heating and granulating using water as a binder, and magnesium stearate is added to obtain 1000 g of a mixture. Then, the mixture is tableted so as to be 1 g per pellet to obtain a tablet-shaped functional food.

### Example 2

### Tablet-Shaped Functional Food

A 250 mg tablet is prepared by mixing EPL (60 mg), lactose (55 mg), corn starch (100 mg), fine crystalline cellulose (20 mg) and magnesium stearate (2.5 mg) and then the mixture is granulated. Fine crystalline cellulose (10 mg) and magnesium stearate (2.5 mg) are then added thereto, and the mixture is tableted to obtain a tablet-shaped functional food.

### Example 3

### Hard Capsule-Shaped Functional Food

Dextrin 140 g is added to 60 g of EPL and mixed homogeneously, and then the above mixture is filled into a hard capsule base material comprising pullulan, vegetable oil, carrageenan and potassium chloride in an amount of each 200 mg per capsule to obtain a hard capsule-shaped functional food.

### Example 4

### Hard Capsule-Shaped Functional Food

Dextrin 140 g is added to 60 g of EPL and mixed homogeneously, and then the above mixture is filled into a hard capsule base material comprising gelatin and glycerin in an amount of each 200 mg per capsule to obtain a hard capsule-shaped functional food.

### Example 5

### Hard Capsule-Shaped Functional Food

A 200 mg capsule is prepared by mixing EPL (60 mg), lactose (60 mg), fine crystalline cellulose (70 mg) and magnesium stearate (5 mg) and then the mixture is granulated. Magnesium stearate (5 mg) is then added thereto, and the whole part is filled into a hard gelatin capsule to obtain a hard capsule-shaped functional food.

### Example 6

### Soft Capsule-Shaped Functional Food

EPL 6 kg is homogeneously suspended in a mixture of safflower oil 23.5 kg, yellow beeswax 2 kg and soybean lecithin 0.5 kg, and then the suspension is encapsulated by a capsule base material comprising carrageenan, starch and glycerin as principal components so that a content liquid per capsule is each 300 mg to obtain an elliptically spherical soft capsule-shaped functional food.

### Example 7

### Soft Capsule-Shaped Functional Food

EPL 6 kg is homogeneously suspended in a mixture of safflower oil 31.5 kg, yellow beeswax 2 kg and soybean lecithin 0.5 kg, and then the suspension is encapsulated by a capsule base material comprising gelatin and glycerin so that a content liquid per capsule is each 400 mg to obtain an elliptical soft capsule-shaped functional food.

### Example 8

### Soft Capsule-Shaped Functional Food

A 400 mg capsule is prepared by mixing EPL (60 mg), a medium chain fatty acid triglyceride (320 mg) and carnauba wax (20 mg) and then filling the mixture the into a soft gelatin capsule to obtain a soft capsule-shaped functional food.

### Example 9

### Granular Functional Food

Dextrin 9 kg is added to EPL powder 1 kg, and the mixture is homogeneously mixed, heated and granulated using water as a binder using a fluidized bed granulating machine to obtain 10 kg of a granulated matter. This granulated matter is filled by means of a stick filling machine so as to be 600 mg per stick to obtain a granular functional food.

### Example 10

### Powdery Functional Food

Dextrin 9 kg is added to EPL powder 1 kg, and the mixture is homogeneously mixed, heated and granulated using water as a binder by means of a fluidized bed granulating machine to obtain 10 kg of a granulated matter. This granulated matter is crushed so that the whole amount passes through a No. 18 sieve and filled using a stick filling machine so as to be 600 mg per stick to obtain a powdery functional food.

### Example 11

### Liquid Functional Food

EPL 6 g is mixed with glucose 0.7 kg, distilled water 5.3 kg and a fragrance 5 g, and the mixture is sterilized by heating and then aseptically filled into a hermetically sealed bottle of 60 mL to obtain a liquid functional food.

### Example 12

### Gelatinous Functional Food

EPL 0.06 kg is mixed with κ-carrageenan 1 kg, liquid sugar of glucose and fructose 17 kg, citric acid 0.36 kg, sodium citrate 0.235 kg and water 82 kg, and then the mixture is heated at 80° C. and filled into a hermetically sealed bottle. Next, it is sterilized by heating according to a conventional method and then cooled down to obtain a gelatinous functional food.

The body weight gain inhibitor, the fat amount gain inhibitor, the medicament for prevention and/or treatment of obesity, the food composition having inhibitory effect on body weight gain and the food composition having inhibitory effect on fat amount gain according to the invention are useful for inhibition of body weight gain, inhibition of fat amount gain and prevention and/or treatment of obesity.

Further, according to the preferred embodiment of the invention, inhibition of body weight gain, inhibition of fat amount gain and prevention and/or treatment of obesity can be carried out without affecting food faddiness and the amount of food intake.

On the other hand, nutritional elements such as fat-soluble vitamins derived from foods necessary for maintaining health are contained as well in an emulsion formed by an ingested fat in the presence of bile acid. Accordingly, when a dose or an intake of ε-polylysine is high, ε-polylysine disturbs absorption of fat and therefore is likely to disturb as well absorption of the above nutritional elements. In the invention, however, a dose or an intake of ε-polylysine is low, and therefore it can be expected that inhibition of body weight gain, inhibition of fat amount gain and prevention and/or treatment of obesity are carried out without disturbing absorption of such nutritional elements necessary for maintaining health.

Although the invention has been described and illustrated with a certain degree of particularity, it is understood that the present disclosure has been made only by way of example, and that numerous changes in the conditions and order of steps can be resorted to by those skilled in the art without departing from the spirit and scope of the invention.

What is claimed is:

1. A method for inhibiting body weight gain comprising administering to mammals in need thereof an effective amount of a body weight gain inhibitor comprising ε-polylysine or a salt thereof, wherein the amount of said ε-polylysine or a salt thereof in said body weight gain inhibitor is from 0.5 mg/kg to 10 mg/kg of body weight per day.

2. The method of claim 1, wherein said patient is clinically obese.

3. The method of claim 1, wherein the degree of polymerization of said ε-polylysine or a salt thereof is in the range of from approximately 20 to approximately 40.

4. A method for inhibiting adipose gain comprising administering to mammals in need thereof an effective amount of a body weight gain inhibitor comprising ε-polylysine or a salt thereof, wherein the amount of said ε-polylysine or a salt thereof in said body weight gain inhibitor is from 0.5 mg/kg to 10 mg/kg of body weight per day.

5. A method for preventing or treating obesity comprising administering to mammals in need thereof an effective amount of a body weight gain inhibitor comprising ε-polylysine or a salt thereof, wherein the amount of said ε-polylysine or a salt thereof in said body weight gain inhibitor is from 0.5 mg/kg to 10 mg/kg of body weight per day.

6. The method of claim 4, wherein a the degree of polymerization of said ε-polylysine or a salt thereof is in the range of from approximately 20 to approximately 40.

7. A method for inhibiting adipose gain comprising administering to mammals an adipose gain inhibitor comprising ε-polylysine or a salt thereof.

8. The method of claim 5, wherein the degree of polymerization of said ε-polylysine or a salt thereof is in the range of from approximately 20 to approximately 40.

9. The method of claim 1, wherein the body weight gain inhibitor is a food composition that is at least one of a nutritional supplement food, a functional food, a health food, a food for specified health use or a food for patients.

10. The method of claim 9, wherein said food composition is in the form of at least one of a tablet, pill, capsule, powder, granule, fine granule, troche or liquid.

11. The method of claim 9, wherein said food composition is at least one of a tablet, a drop, a candy, a jelly, a drink, a cookie, a cracker, a biscuit, a chocolate, margarine or a chewing gum.

12. The method of claim 9, wherein the amount of said ε-polylysine or a salt thereof in said food composition is from approximately 0.05% by weight to approximately 5% by weight based upon the fat content of said food composition.
